(19) United States
(12) Patent Application Publication
(10) Pub. No.: US 2013/0052247 A1
(43) Pub. Date: Feb. 28, 2013

(54) FLEXIBLE ARTICLE FOR NAILS WITH AN IMPROVED ADHESIVE LAYER

(71) Applicants: Guillaume Kergosien, Chaville (FR); Carl Riachi, Paris (FR)

(72) Inventors: Guillaume Kergosien, Chaville (FR); Carl Riachi, Paris (FR)

(73) Assignee: L'OREAL, Paris (FR)

(21) Appl. No.: 13/696,670

(22) PCT Filed: May 11, 2011
(86) PCT No.: PCT/IB2011/052065

§ 371 Date: Nov. 7, 2012

Related U.S. Application Data
(60) Provisional application No. 61/344,090, filed on May 21, 2010.

(30) Foreign Application Priority Data
May 11, 2010 (FR) 1053690

(51) Int. Cl. A61K 8/90; A61Q 3/02; A45D 31/00

(52) U.S. Cl. 424/401; 424/61

(57) ABSTRACT

The present invention relates to a flexible article for application to the nails and/or false nails so as to make them up and/or care for them, the article comprising at least one adhesive layer, and being characterized in that said adhesive layer comprises at least two block copolymers, a tackifier resin, and a plasticizer.

The present invention relates to a flexible article for application to the nails and/or to false nails so as to make them up and/or care for them.

Conventionally, nails or false nails are made up using liquid makeup compositions, still generally known as nail polish. Such nail polish is applied to the surface of the nail to be made up, generally in the form of superposed layers, allowing an intermediate drying step between each application of nail polish.

Firstly, applying it requires a certain amount of time.

Furthermore, the more or less liquid formulations of the nail polish tend, during application, to creep over the bordering or peripheral surrounds of the surface to be made up.

In addition, after about 3 to 5 days, the nail polish is generally observed to deteriorate, in particular as a result of chipping, and a reduction in shine.

The user must thus proceed to a step of removing said polish prior to applying a new layer of polish.

Consequently, such a makeup method is not entirely satisfactory.

Another drawback consists in that conventional nail polish formulations generally involve the use of volatile solvents that generate a disagreeable odor during application.

An alternative consists in developing nail polish compositions based on a dispersion of polymers in an aqueous phase, which is thus satisfactory as regards olfactory properties. Unfortunately, the method of application is still restrictive, and the corresponding polishes also turn out to have insufficient staying power.

Still another alternative consists in developing adjustable adhesive patches in the form of adhesive laminates or flexible articles comprising an adhesive layer, and, where appropriate, a polymeric layer. Such alternatives are described in particular in U.S. Pat. No. 4,903,840, US 2005/255061, U.S. Pat. No. 5,415,903, WO 2005/112874, and WO 2009/097517.

However, such patches present insufficient deformability and/or stretchability. The adhesive layers of these various articles also generally present hold on the nail that is not entirely satisfactory.

The present invention specifically seeks to propose a method of making up and/or caring for the nails or false nails that does not have the above-mentioned drawbacks, and that presents improved staying power and improved flexibility or deformability, in particular so as to enable the article to be applied in rapid and easy manner to the nails and/or false nails.

First exemplary embodiments of the present invention thus provide a flexible article for application to the nails and/or false nails so as to make them up and/or care for them, the article being in particular in the form of an adhesive laminate comprising at least one adhesive layer, and being characterized in that said adhesive layer comprises at least:

two block copolymers;

a tackifier resin having a number-average molecular weight that is less than or equal to 10000 grams per mole (g/mol) selected from rosin, rosin derivatives, hydrocarbon resins, and mixtures thereof; and

a plasticizer.

In unexpected manner, the inventors have observed not only that such a flexible article presents greater hold on the nails, but also that it is capable of being applied to the nails quickly and easily because of its properties of flexibility and deformability.

In other exemplary embodiments, the present invention provides a method of making up and/or caring for the nails and/or false nails, the method comprising at least one step consisting in applying at least one flexible article of the invention to a natural nail and/or a false nail.

In exemplary embodiments, the flexible article of the present invention may present various shapes, such as a star, a square, a circle, etc.

In a variant, the article may further include a polymeric layer or a succession of polymeric layers.

The Flexible Article

In general, the article of the present invention is in the form of a film or a laminate.

In the meaning of the present invention, the term “flexible” means a flexibility of the film that is sufficient, i.e. favoring mechanical deformations of the stretch-type in order to adjust it to the surface of a nail.

In addition, the term “flexible” should also be understood to mean capable of deforming non-elastically, in such a manner as to match the shape of the more or less convex profile of the nail.

This deformability is especially characterized by the deformation-at-break parameter, εᵣ, discussed below.

In particular, the article of the invention differs from a false-nail type article that is characterized by stiffness that is incompatible with such mechanical deformation.

Another difference between the article of the invention and a false nail lies in the fact that this article is sensitive to polar organic solvents of the acetone, ester and/or lower alcohol type, such as alkyl acetates, in particular ethyl acetate.

The article of the invention may easily be removed by means of a conventional remover or solvent, as opposed to a false nail that has to be taken off.

The invention thus relates to an article that can be removed by means of a solvent selected from acetone, alkyl acetates such as ethyl acetate, and mixtures thereof.

In all these respects, the flexible article of the invention differs firstly from conventional liquid nail-polish type compositions because of its dry structure, and secondly from solid false-nail type products by the fact that, prior to application, said article is mechanically deformable and is suitable for removing with remover, unlike a false nail that is attached directly to the nail, then filed so as to fit, and subsequently removed directly from the nail so as to return the nail to its normal state.

More particularly, the flexible article of the invention is different from conventional liquid nail-polish type compositions. Indeed its dry structure is obtained prior to its application on the nails or false-nails whereas conventional liquid nail-polish type compositions display a film-forming structure only after application of said compositions on the nails or false-nails, and further after a step of drying.

The article of the invention may be used for make-up purposes, in which circumstance it may include coloring substances, or it may be used for protective purposes for protecting a film of polish. In this alternative, the polymeric layer, when one is present, is generally transparent.

In particular, the flexible article may further include a protective membrane in contact with the first face of the adhesive layer and for removing prior to putting the article into place on the nail. Preferably, the face of the protective membrane in contact with the first face of the adhesive layer is covered with a non-stick material, in particular a silicone material.

In particular exemplary embodiments, both faces of the article of the invention are coated with identical or different removable membranes.

In exemplary embodiments, the flexible article of the present invention presents thickness lying in the range 6 micrometers (μm) to 1 millimeter (mm), in particular in the range 10 μm to 500 μm, and even more particularly in the range 50 μm to 200 μm.

The thickness in question should be understood as the thickness prior to application to the nail of the entire indissociable structure of one or more layers, in particular including the adhesive layer.

In particular, the thickness means the thickness of all of the layers constituting the flexible article, such as all of the polymeric and adhesive layers, for example.

After drying, the adhesive layer of a flexible article of the invention is generally in the form of a layer having thickness that is greater than 30 μm, preferably greater than 40 μm.

Preferably, the adhesive layer has a thickness lying in the range 30 μm to 100 μm, in particular in the range 40 μm to 95 μm, preferably in the range 45 μm to 90 μm, and better in the range 50 μm to 85 μm.

Preferably, the thickness of the adhesive layer is greater than the thickness of the polymeric layer.

Preferably, the thickness of the adhesive layer is greater than half the thickness of said polymeric layer.

As described above, in preferred exemplary embodiments, a polymeric layer of an article of the invention may comprise a first layer and a second layer that is adjacent to said first layer.

In this particular embodiment, the thickness of the second layer is thus preferably less than 20 μm, and more particularly less than 10 μm.

Advantageously, the ratio between the thickness of the first layer and the thickness of the second layer preferably varies in the range 3 to 50, preferably in the range 4 to 25, and more preferably in the range 5 to 20.

In contrast, any structure that is removably fastened to the flexible article, in particular a protective membrane on one or the other of the faces of the article, in particular a silicone membrane on the adhesive face of the article, is not taken into account when measuring the thickness.

The flexible article, and in particular the excess, may be pre-cut or cut out to the desired shape and form, before or after application thereof, by using small scissors, or nail clippers, or by scratching the film.

The flexible article of the invention is in particular in the form of a non-liquid film that may be characterized by a high dry extract. The quantity of dry material in the article in the dry state, i.e. once applied to the nail or the false nails, is greater than or equal to 95% by weight relative to the total weight of the article. In other words, the quantity of volatile solvent is less than or equal to 5% by weight relative to the total weight of the article.

Preferably, the quantity of dry matter, usually termed the “dry extract” of the articles of the invention, is measured by heating a sample using infrared radiation with a wavelength in the range 2 μm to 3.5 μm. Substances contained in said films that have a high vapor pressure evaporate off under the effect of this radiation. Measuring the loss of mass of the sample allows the dry extract of the article to be determined. Said measurements are made using a commercial LP16 infrared desiccator from Mettler. That technique is fully described in the documentation furnished by Mettler accompanying the apparatus.

The measurement protocol is as follows:

About 10 grams (g) of sample of an article is deposited in a metal cup. After being introduced into a dessicator, it is subjected to a temperature of 120° C. for one hour. The moist mass of the sample corresponding to the initial mass, and the dry mass of the sample corresponding to the mass after exposure to radiation, are measured using a precision balance.

The dry matter content is calculated as follows:

Dry extract=100×(dry mass/moist mass).

Water Take-Up

Advantageously, the article of the invention is characterized in the dry state by a water take-up at 25° C. of 20% or less, in particular 16% or less, and more particularly less than 10%.

In the present application, the term “water take-up” denotes the percentage of water absorbed by the article after immersion in water for 60 minutes (min) at 25° C. (ambient temperature). The water take-up is measured using pieces of about 1 centimeter (cm) cut from the dry article. They are weighed (measurement of mass M1), then immersed in water for 60 min; after immersion, the piece of film is wiped to eliminate excess surface water then weighed (measurement of mass M2). The difference, M2−M1, corresponds to the quantity of water absorbed by the film.

The water take-up is equal to [(M2−M1)/M1]×100 and is expressed as the percentage by weight relative to the weight of the film.

Storage Modulus E′

Furthermore, the article of the invention is advantageously a film having a storage modulus E′ of 1 megapascal (MPa) or more, in particular 1 MPa to 5000 MPa, more particularly 5 MPa or more, in particular 5 MPa to 1000 Mpa, and still more particularly 10 MPa or more, for example 10 MPa to 500 MPa at a temperature of 30° C. and a frequency of 0.1 Hertz (Hz).

The storage modulus is measured by DMTA (Dynamic and Mechanical Temperature Analysis).

The viscoelastic tests are carried out with a DMTA apparatus from Polymer TA Instruments (DMA2980 model) on a sample of the article. Specimens are cut out (for example using a punch). They have a typical thickness of about 150 μm, a width of 5 mm to 10 mm, and a useful length of about 10 mm to 15 mm.

The measurements are carried out at a constant temperature of 30° C.

The sample is placed under tension and subjected to small deformations (for example a sinusoidal displacement of ±8 μm) during a frequency scan, the frequency being from 0.1 Hz to 20 Hz. Thus, the working region is linear, with small deformations.

Said measurements allow the complex modulus E*=E′+iE″ of the test composition film to be determined, E′ being the storage modulus and E″ the “lossy” modulus.

Deformation and/or Energy at Break

Advantageously, the articles of the invention have a deformation at break Eᵣ of 5% or more, in particular 5% to 500%, more preferably 15% or more, especially 15% to 400%, and/or an energy at break per unit volume Wᵣ of 0.2 joules/cubic centimeter (J/cm³) or more, in particular 0.2 J/cm³ to 100 J/cm³, preferably more than 1 J/cm³, in particular 1 J/cm³ to 50 J/cm³.

The deformation at break and the energy at break per unit volume are determined by tensile tests carried out on a cross-linked film about 200 μm thick.

To carry out these tests, the article is cut into dumb-bell shaped test specimens with a useful length of 33±1 mm and a useful width of 6 mm.

The section (S) of the specimen is thus defined as:

S=width×thickness (cm²); this section is used for the stress calculation.

The tests are carried out, for example, using a commercial tensile test apparatus sold under the trade name Lloyd® LR5K. The measurements are carried out at ambient temperature (20° C.)

The specimens are stretched at a displacement rate of 33 millimeters per minute (mm/min), corresponding to an extension rate of 100% per minute.

Thus, a displacement rate is imposed and the extension ΔL of the specimen and the force F necessary to impose said extension are measured simultaneously.

These data, ΔL and F, are used to determine the stress σ and deformation ε parameters. A stress curve of σ=(F/S) is obtained as a function of the deformation ε=(ΔL/L₀)×100, the test being carried out until the sample breaks, L₀ being the initial length of the sample. The deformation at break εᵣ is the maximum deformation of the sample before the break point (as a %). The energy at break per unit volume, Wᵣ in J/cm², is defined as the area beneath the stress/deformation curve, i.e.:

$$Wr = {\int }_{0}^{{ɛ}_{r}} σ \cdot ɛ \cdot d ɛ$$

The Adhesive Layer

An article of the invention has an adhesive outer face. Such an adhesive face is generally obtained by means of the presence of at least one adhesive layer, the adhesive face being characterized in that the adhesive layer comprises at least two block copolymers, a tackifier resin, and a plasticizer.

The combination of at least two block copolymers, a tackifier resin, and a plasticizer should present a certain adhesive power as defined by its viscoelastic properties.

The viscoelastic properties of a material are conventionally defined by two characteristic values that are as follows:

the elastic modulus that represents the elastic behavior of the material for a given frequency and that is conventionally denoted G′;

the viscous modulus that represents the viscous behavior of the material for a given frequency that is conventionally denoted G″.

These magnitudes are defined in the “Handbook of Pressure Sensitive Adhesive Technology” 3rd edition, D. Satas, chap. 9, pp. 155 to 157.

Preferably, adhesive materials that are suitable for use in the present invention have viscoelastic properties that are measured at a reference temperature of 35° C. and in a certain frequency range.

In the case of adhesive materials in the form of a solution or dispersion of polymer in a volatile solvent (such as water, a short chain ester, a short chain alcohol, acetone, etc), the viscoelastic properties of the material are measured under conditions under which it has a volatile solvent content of less than 30%, in particular a volatile solvent content of less than 20%.

In particular, the elastic modulus of the material is measured at three different frequencies:

at low frequency, i.e. 2×10⁻² Hz;

at an intermediate frequency, i.e. 0.2 Hz;

at high frequency, i.e. at 2 Hz; and

the viscous modulus at the frequency of 0.2 Hz.

These measurements allow the change of adhesive power of the adhesive material over time to be measured.

These viscoelastic properties are measured during dynamic tests under low amplitude sinusoidal stresses (small deformations) carried out at 35° C. over a frequency range of 2×10⁻² to 20 Hz using a “Haake RS50®” type rheometer under tension/shear stress, for example in cone/plane geometry (for example with a cone angle of) 1°).

Advantageously, said adhesive material satisfies the following conditions:

G′(2 Hz, 35° C.)≧10³ Pa; and

G′(35° C.)≦10⁸ Pa, in particular G′(35° C.)≦10⁷ Pa;

G′(2×10⁻² Hz, 35° C.)≦3×10⁵ Pa;

in which:

G′(2 Hz, 35° C.) is the elastic shear modulus of said adhesive material, measured at a frequency of 2 Hz and at a temperature of 35° C.;

G′(35° C.) is the elastic shear modulus of said adhesive material, measured at a temperature of 35° C., for any frequency in the range 2×10⁻² Hz to 2 Hz;

G′(2×10⁻² Hz, 35° C.) is the elastic shear modulus of said adhesive material, measured at a frequency of 2×10⁻² Hz and at a temperature of 35° C.

In a particular form of the invention, the adhesive material also satisfies the following condition:

G″/G′(0.2 Hz, 35° C.)≧0.35.

in which:

G″(0.2 Hz, 35° C.) is the viscous shear modulus of said adhesive material, measured at a frequency of 0.2 Hz and at a temperature of 35° C.;

G′(0.2 Hz, 35° C.) is the elastic shear modulus of said adhesive material, measured at a frequency of 0.2 Hz and at a temperature of 35° C.

In a particular form of the invention:

G′(2 Hz, 35° C.)≧5×10³ Pa, and in particular, G′(2 Hz, 35° C.)≧10⁴ Pa.

In a further particular form of the invention:

G′(2×10⁻² Hz, 35° C.)≦5×10⁴ Pa.

In particular, the adhesive materials of the invention satisfy the following four conditions:

G′(2 Hz, 35° C.)≧10⁴ Pa; and

G′(35° C.)≦10⁸ Pa, in particular G′(35° C.)≦10⁷ Pa;

G′(2×10⁻² Hz, 35° C.)≦5×10⁴ Pa; and

G″/G′(0.2 Hz, 35° C.)≧0.35.

Block Copolymers

A flexible article of the invention comprises at least one adhesive layer that comprises at least two block copolymers.

In the meaning of the present invention, the term “copolymer” means a polymer resulting from at least two monomers types.

The term “block” or “sequence” copolymer means a polymer comprising at least two distinct sequences or blocks.

The copolymers may present a linear structure or a non-linear structure such as a copolymer having a branched, radial, or star structure, for example.

The block copolymers of the invention may be statistical or alternating.

Preferably, the block copolymers of the invention are selected from amorphous olefin copolymers or copolymers having controlled and moderate crystallization, and preferably from amorphous copolymers.

The term “amorphous copolymer” means a polymer that does not have a crystalline form.

The olefin copolymer may also be film-forming, i.e. it is capable of forming a film while being applied to the skin.

In the meaning of the present application, the term olefin copolymer means any copolymer formed by the polymerization of at least one olefin and another additional monomer that is different from said olefin, such as a styrene monomer, for example.

In particular, the olefin may be a monomer with an ethylenically unsaturated bond.

As an example of olefins, mention may be made of ethylene carbide monomers, in particular having one or two ethylenically unsaturated bonds (or alkenes), having 2 to 5 carbon atoms such as ethylene, propylene, butadiene, isoprene.

In exemplary embodiments, at least one of the block copolymers of the invention includes at least one styrene block.

In other exemplary embodiments, the block copolymers result from the polymerization of at least one olefin and at least one styrene block.

In still other exemplary embodiments, at least one of the block copolymers of the invention includes at least one block including at least one pattern selected from butadiene, ethylene, propylene, butylene, isoprene, or a mixture thereof.

In still other exemplary embodiments, each of the block copolymers of the invention includes at least one styrene block.

In still other exemplary embodiments, each of the block copolymers of the invention includes at least one block including at least one pattern selected from butadiene, ethylene, propylene, butylene, isoprene, or a mixture thereof.

In still other exemplary embodiments, each of the block copolymers of the invention includes at least one styrene block and at least one block including at least one pattern selected from butadiene, ethylene, propylene, butylene, isoprene, or a mixture thereof.

Finally, in still other exemplary embodiments, each of the block copolymers of the invention includes at least one styrene block and at least one block including at least one isoprene pattern.

Advantageously, the styrene content in a block copolymer of the invention is less than 35%, preferably less than 25%.

Thus, in an embodiment, the article is such that the block copolymers are selected from sequenced copolymers including at least one styrene block, in particular present at a content of less than 35%, preferably less than 25%, and at least one block including at least one pattern selected from butadiene, ethylene, propylene, butylene, isoprene, or a mixture thereof.

In the meaning of the present invention, a block copolymer of the invention may be selected from a diblock, triblock, multiblock, radial, or star copolymer.

In particular exemplary embodiments, one of the two block copolymers is selected from a diblock copolymer and the other is selected from a triblock-, multiblock-, radial-, or star-type copolymer.

Advantageously, the triblock-, multiblock-, radial-, or star-type copolymer is selected from the triblock-type copolymers.

Mixtures of block copolymers of the invention may in particular be selected from:

mixtures of styrene-isoprene-styrene linear triblock copolymers and of styrene-isoprene diblock copolymers, in particular available from the supplier KRATON under the references KRATON D1107P, KRATON D1111K, KRATON D1113P, KRATON D1161K, and KRATON D1161P, and in particular also available from the supplier DEXCO POLYMERS under the reference VECTOR 4113;

mixtures of styrene and isoprene block copolymers having radial structure and of styrene-isoprene diblock copolymers, in particular available from the supplier KRATON under the reference KRATON D1126P, and in particular also available from the supplier DEXCO POLYMERS under the reference VECTOR 4230;

mixtures of styrene-butadiene/isoprene-styrene linear triblock copolymers and of styrene-butadiene/isoprene diblock copolymers, in particular available from the supplier KRATON under the references KRATON D1170B and KRATON D1171P;

mixtures of styrene-ethylene/butadiene-styrene linear triblock copolymers and of styrene-ethylene/butadiene diblock copolymers, in particular available from the supplier KRATON under the reference KRATON G1657;

and mixtures thereof.

In advantageous manner, the two block copolymers are present at a content lying in the range 1% to 70% by weight relative to the total weight of the adhesive layer, preferably in the range 5% to 50% by weight, and more particularly in the range 10% to 30% by weight.

Advantageously, the weight ratio “triblock, multiblock, radial, or star copolymer”/“diblock copolymer” is less than 7, and preferably less than 5.

Tackifier Resin

A flexible article of the invention includes at least one adhesive layer that includes at least one tackifier resin.

In the meaning of the invention, a tackifier resin generally has a number average molecular weight that is less than or equal to 10000 g/mol, in particular lying in the range 250 g/mol to 10000 g/mol, preferably less than or equal to 5000 g/mol, in particular lying in the range 250 g/mol to 5000 g/mol, better less than or equal to 2000 g/mol, in particular lying in the range 250 g/mol to 2000 g/mol, or better still less than or equal to 1000 g/mol, in particular lying in the range 250 g/mol to 1000 g/mol.

The number average molecular weights (Mn) may be determined by liquid gel permeation chromatography (solvent THF, calibration curve established with linear polystyrene standards, refractometric detector).

A tackifier resin of the invention is advantageously a resin as described in the Handbook of Pressure Sensitive Adhesive, edited by Donatas Satas, 3rd ed., 1989, pp. 609-619.

The resin of the composition of the invention is selected from rosin, rosin derivatives, hydrocarbon resins, in particular hydrogenated resins, and mixtures thereof. Preferably, the resin of the composition of the invention is selected from hydrocarbon resins.

Rosin is a mixture mainly comprising organic acids called rosin acids (mainly acids of the abietic type and of the pimaric type).

Three types of rosin exist: gum rosin obtained by incision on live trees, wood rosin that is extracted from pine logs or wood, and tall oil rosin that is obtained from a by-product deriving from the production of paper.

Rosin derivatives may in particular result from polymerization, hydrogenation, and/or esterification of rosin acids. Rosin derivatives may thus be selected from rosin esters, in particular resulting from polyhydric alcohols such as ethylene glycol, glycerol, pentaerhytritol, and more particularly resulting from glycerol and from pentaerhytritol. By way of example, mention may be made of rosin esters sold under the reference FORAL 85, PENTALYN H, and STAYBELITE ESTER 10 by the supplier HERCULES; SYLVATAC 95 and ZONESTER 85 by the supplier ARIZONA CHEMICAL; or UNIREZ 3013 by the supplier UNION CAMP.

The term “hydrocarbon tackifier resin” means an olefin polymer or copolymer or an aromatic hydrocarbon monomer polymer or copolymer.

Said polymer may be hydrogenated, partially hydrogenated, or non-hydrogenated. Preferably, the tackifier resin is hydrogenated.

The tackifier resin preferably presents a softening point that is less than 120° C., preferably less than 110° C.

In the context of the present invention, the softening point is measured using the ring-and-ball method in accordance with the ASTM D36 Standard. To do this, use is made of an automatic NBA 440 tester available from Normalab. The fluid used for the measurement is glycerine.

Hydrocarbon tackifier resins of the invention may in particular be selected from polymers that may, depending on the type of monomer that they contain, be classified as:

indene hydrocarbon resins such as the resins derived from the polymerization of indene monomer in the greater proportion, and of a monomer selected from styrene, methylindene, methylstyrene, and mixtures thereof in the lesser proportion. These resins may possibly be hydrogenated. These resins may present a molecular weight lying in the range 290 g/mol to 1150 g/mol.

Examples of indene resins that may be mentioned are those sold under the reference NORSOLENE S95, NORSOLENE 5105, NORSOLENE 5115 by the Supplier Cray Valley, or hydrogenated indene/methylstyrene/styrene copolymers sold under the trade name “REGALITE” by the supplier Eastman Chemical, in particular REGALITE C6100, REGALITE C6100L, REGALITE R1090, REGALITE R1100, REGALITE R7100, REGALITE R9100, REGALITE 51100, REGALITE 55100, or under the trade name ARKON P-90, ARKON P-100, ARKON P-115, ARKON M-90, ARKON M-100, ARKON M-115 by the supplier Arakawa.

aliphatic pentadiene resins such as that derived from the polymerization mainly of 1,3-pentanediene (trans or cis piperylene) and of a minor monomer selected from isoprene, butene, 2-methyl-2-butene, pentene, 1,4-pentadiene and mixtures thereof. These resins may present a molecular weight lying in the range 1000 g/mol to 2500 g/mol.

By way of example, such 1,3-pentadiene resins are sold under the references PICCOTAC 95 by the supplier Eastman Chemical, ESCOREZ 1102, ESCOREZ 1304, ESCOREZ 1310LC, ESCOREZ 1315 by the supplier Exxon Chemicals, WINGTACK 95 by the supplier Cray Valley;

mixed pentadiene and indene resins that are derived from the polymerization of a mixture of pentadiene and indene monomers such as those described above, such as for example the resins sold under the reference ESCOREZ 2101, ESCOREZ 2105, ESCOREZ 2173, ESCOREZ 2184, ESCOREZ 2203LC, ESCOREZ 2394, ESCOREZ 2510 by the supplier Exxon Chemicals, NORSOLENE A 100 by the supplier Cray Valley, the resins sold under the reference WINGTACK 86, WINGTACK EXTRA and WINGTACK PLUS by the supplier Cray Valley;

diene resins from cyclopentadiene dimers, such as those derived from the polymerization of a first monomer selected from indene and styrene, and a second monomer selected from dimers of cyclopentadiene such as dicyclopentadiene, methyldicyclopentadiene, other dimers of pentadiene, and mixtures thereof.

These resins generally present a molecular weight lying in the range 500 g/mol to 800 g/mol, such as for example those sold under the reference ESCOREZ 5380, ESCOREZ 5300, ESCOREZ 5400, ESCOREZ 5415, ESCOREZ 5490, ESCOREZ 5600, ESCOREZ 5615, ESCOREZ 5690, by the supplier Exxon Mobil Chem., and the resins SUKOREZ SU-90, SUKOREZ SU-100, SUKOREZ SU-110, SUKOREZ SU-100S, SUKOREZ SU-200, SUKOREZ SU-210, SUKOREZ SU-490, SUKOREZ SU-400, by the supplier Kolon;

diene resins from isoprene dimers such as the terpene resins derived from the polymerization of at least one monomer selected from α-pinene, β-pinene, limonene, styrene, and mixtures thereof, and in particular polyterpene, and the terpene resins derived from styrene;

These resins may present a molecular weight lying in the range 300 g/mol to 2000 g/mol. By way of example, such resins are sold under the trade name PICCOLYTE A115 by the supplier Hercules, ZONAREZ 7100 or ZONATAC 105 LITE by the supplier ARIZONA Chem.;

hydrogenated resins derived from the polymerization of pentadiene such as those sold under the trade name EASTOTAC H-100E, EASTOTAC H-115E, EASTOTAC C-100L, EASTOTAC C-115L, EASTOTAC H-100L, EASTOTAC H-115L, EASTOTAC C-100R, EASTOTAC C-115R, EASTOTAC H-100R, EASTOTAC H-115R, EASTOTAC C-100W, EASTOTAC C-115W, EASTOTAC H-100W, EASTOTAC H-115W, by the supplier Eastman Chemical Co.; and

mixtures thereof.

In particular exemplary embodiments, the resin is selected from indene hydrocarbon resins sold under the trade name NORSOLENE 595, NORSOLENE 5105, NORSOLENE 5115 by the supplier Cray Valley, under the trade name “REGALITE” by the supplier Eastman Chemical, in particular REGALITE C6100, REGALITE C6100L, REGALITE R1090, REGALITE R1100, REGALITE R7100, REGALITE R9100, REGALITE 51100, REGALITE 55100, or under the trade name ARKON P-90, ARKON P-100, ARKON P-115, ARKON M-90, ARKON M-100, ARKON M-115 by the supplier Arakawa.

In advantageous manner, the layer that is adhesive in the meaning of the present invention contains tackifier resin(s) at a content lying in the range 10% to 95% by weight relative to the total weight of said adhesive layer, preferably in the range 20% to 90% by weight, and more particularly in the range 40% to 80% by weight.

Plasticizer

A flexible article of the invention includes at least one adhesive layer that includes at least one plasticizer.

In particular, it is possible to use, alone or as a mixture, the usual plasticizers such as: glycols and their derivatives such as diethylene glycol ethyl ether, diethylene glycol methyl ether, diethylene glycol butyl ether or diethylene glycol hexyl ether, ethylene glycol ethyl ether, ethylene glycol butyl ether, ethylene glycol hexyl ether; glycol esters; propylene glycol derivatives, in particular propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether and diethylene glycol methyl ether, propylene glycol butyl ether; esters, in particular of carboxylic acids such as citrates, in particular triethyl citrate, tributyl citrate, triethyl acetyl citrate, tributyl acetyl citrate, 2-triethylhexyl acetyl citrate; phthalates, in particular diethyl phthalate, dibutyl phthalate, dioctyl phthalate, dipentyl phthalate, dimethoxyethyl phthalate; phosphates, in particular tricresyl phosphate, tributyl phosphate, triphenyl phosphate, tributoxyethyl phosphate; tartrates, in particular dibutyl tartrate; adipates; carbonates; sebacates; benzyl benzoate; butyl acetylricinoleate; glyceryl acetylricinoleate; butyl glycolate; camphor; glycerol triacetate; N-ethyl-o,p-toluenesulfonamide; oxyethylenated derivatives such as oxyethylenated oils, in particular vegetable oils such as castor oil; silicone oils; hydrocarbon oils; and mixtures thereof.

In advantageous manner, the plasticizer of the invention is selected from hydrocarbon oils, in particular from optionally-hydrogenated polyisobutenes.

By way of example, such plasticizers are sold under the trade name PARLEAM® by the supplier NOF CORPORATION.

In particular exemplary embodiments, the adhesive layer(s) of the invention may contain plasticizer(s) at a content lying in the range 5% to 40% by weight relative to the total weight of said adhesive layer(s), in particular in the range 10% to 35% by weight, and more particularly in the range 15% to 30% by weight.

The Polymeric Layer

In addition to the adhesive layer, an article of the present invention may include at least one polymeric layer.

In the present invention, the polymeric layer may be a single polymeric layer. However, the polymeric layer may also be a combination or an arrangement of two or more identical or different polymeric layers that together form the polymeric layer.

In preferred exemplary embodiments, a polymeric layer comprises a first layer and a second layer that is adjacent to said first layer.

Advantageously, the second layer may be substantially transparent.

In the present invention, the polymeric layer (or sub-layer(s)) includes at least one film-forming polymer. The term “film-forming polymer” means a polymer that can form, by itself or in the presence of an additional film-forming agent, a continuous film that adheres to a surface, in particular to keratinous materials.

In preferred exemplary embodiments, the polymeric layer (or sub-layer(s)) may further include at least one film-forming co-agent and/or at least one film-modifying agent.

In particularly preferred exemplary embodiments, the article of the invention comprising a polymeric layer has Persoz hardness that is less than 50 seconds (s), preferably less than 40 s, more particularly less than 35 s, and more particularly less than 30 s, and/or resistance to abrasion corresponding to a weight loss that is less than 50 milligrams (mg), preferably less than 40 mg, better less than 30 mg, and more particularly less than 20 mg, the polymeric arrangement being configured in such a manner that, when the article is also substantially exempt of solvents, the elongation at rupture of the article is greater than 30%, preferably greater than 40%, more particularly greater than 50%, and even more particularly greater than 60%.

The Persoz hardness may be determined by any appropriate method known to the person skilled in the art.

For example, the Persoz hardness may be determined using a Persoz pendulum at a defined temperature, e.g. 30° C., and at a defined humidity rate, e.g. at 50% relative humidity (RH).

An appropriate protocol relating thereto is described below.

The protective layers of the article are removed.

The adhesive side of the article is applied to a glass plate and is then dried at 30° C. for 23 hours (h), then for one additional hour in the environment of the pendulum.

The Persoz pendulum is then positioned above the glass plate. The time required by the Persoz pendulum to oscillate from an amplitude of 12° to an amplitude of 4° is then determined. A plurality of measurements, e.g. ten measurements, and thus taken so as to establish an average of the calculated results.

In preferred exemplary embodiments of the present invention, the adhesive layer is in direct contact with the polymeric layer.

Film-Forming Polymer

The term “film-forming polymer” or “film-forming agent”, that are used without distinction in the present description, should, in the meaning of the present invention, be understood as any polymer or resin that leaves a film on the substrate to which it is applied, e.g. after a solvent accompanying said polymer or said film-forming agent has evaporated from the substrate, has been absorbed into it, and/or has dissipated on it. In particular, the term “film-forming polymer” means a polymer that can form, by itself or in the presence of an additional film-forming agent, a continuous film that adheres to a surface, in particular to keratinous materials.

In preferred exemplary embodiments, the film-forming agent is a polymer having a molecular weight that is greater than the critical entanglement weight. In particular, the polymer may be solid at ambient temperature. In the context of the present invention, the “critical entanglement weight” may be calculated using the modulus G′ at the plateau zone, as described in the extract from “Viscoelastic Properties of Polymers”, 2^{ème} edition, 1970—John D. Ferry—John Wiley & Sons, Inc., pages 403-404:

“In the conceptual scheme of entanglement coupling, the most important parameter is the average molecular weight between the coupling loci, Mₑ, or the average number of chain atoms, jPₑ=jMₑ/M₀, where j is the number of chain atoms per monomer unit and M₀ the molecular weight of the monomer. [ . . . ] There are several methods for estimating this value.

If G′ were totally independent of frequency in the plateau zone [ . . . ], its value here, which may be called G_{eN}⁰, the pseudo-equilibrium modulus of the entanglement network, could be related by analogy with [ . . . ] the density of entanglement network strands νₑ:

G_{eN}⁰=g_{N}νₑRT=g_{N}ρRT/Mₑ=1/J_{eN}⁰

where g_{N} is a numerical factor close to unity (not necessarily the same as g in the earlier equation) and p is the density. The value of G_{eN}⁰ could similarly be obtained from the plateau zone of the relaxation module; or its reciprocal, Je_{N}⁰ [ . . . ] from the plateau of the creep compliance or the dynamic storage compliance”.

Numerous values estimated in this way have been indicated in the literature, examples of which are give in Table 1 below.

TABLE 1

| | | | | | | | |
|---|---|---|---|---|---|---|---|
| | Temp | Log | | | | | |
| Polymer | ° C. | J_{eN}⁰ | Mₑ | J | M₀ | jPₑ | Ref |

Methacrylate polymers

| | | | | | | | |
|---|---|---|---|---|---|---|---|
| Methyl (atactic) | 110 | −6.79 | 5900 | 2 | 100 | 59 | 15 |
| 2-ethyl butyl | 100 | −6.16 | 21400 | 2 | 170 | 130 | 16 |
| rubber | | | | | | | |
| n-hexyl | 100 | −5.94 | 33900 | 2 | 170 | 200 | 17 |
| n-octyl | 100 | −5.52 | 87000 | 2 | 198 | 440 | 18 |

Rubbers

| | | | | | | | |
|---|---|---|---|---|---|---|---|
| Hevea rubbers | 25 | −6.59 | 5750 | 4 | 68 | 340 | 1 |
| 1,4-polybutadiene | 25 | −7.06 | 1900 | 4 | 54 | 140 | 2 |
| 1,4-polybutadiene, | 25 | −6.88 | 2950 | 4 | 54 | 220 | 19 |
| cis | | | | | | | |
| 1,2-polybutadiene | 25 | −6.79 | 3550 | 2 | 54 | 130 | 1 |
| Styrene-butadiene | 25 | −6.89 | 3000 | 4 | 65.5 | 180 | 20 |
| copolymer | | | | | | | |
| Butyl rubber | 25 | −6.46 | 8500 | 2 | 56 | 300 | 19 |
| Ethylene-propylene | 25 | −7.10 | 1660 | 2 | 34.3 | 100 | 21 |
| copolymer | | | | | | | |

General

| | | | | | | | |
|---|---|---|---|---|---|---|---|
| Polyisobutylene | 25 | −6.40 | 8900 | 2 | 56 | 320 | 22 |
| Polydimethyl | 25 | −6.47 | 8100 | 2 | 74 | 220 | 23 |
| siloxane | | | | | | | |
| Polystyrene | 160 | −6.30 | 18100 | 2 | 104 | 350 | 3 |

The list of chemical substances from which suitable polymers or copolymers may be selected include (but is not limited to) polyolefins, (polyethylene, polypropylene, polyisobutylene, etc.), polyvinyl chloride, polyvinyl acetate and derivatives thereof, styrenics, polyacrylics, polyesters, polyamides, polyimides, polyoxyalkylenes, fluorinated polymers, cellulosics, aromatic skeleton polymers, polycarbonates, aromatics polysulfones, polyphenylene sulfides (PPS), polyphenylene ethers, polyetherimides, aromatics polyamides, polyamide-imides, polyaryletherketones, polyurethanes, silicone rubbers, phenolic polymers, formophenolic polymers, epoxy compounds, and mixtures thereof.

In preferred exemplary embodiments, the polymeric layer includes at least one film-forming agent that is capable of being cross-linked.

In these exemplary embodiments, any cross-linking means is acceptable such as chemical cross-linking, ionic cross-linking (complexing), and physical cross-linking (with H bond and/or phase segregation), for example.

Preferably, Such a polymer has at least one Tg (glass transition temperature) less than ambient temperature.

Examples of suitable polymers that may be mentioned include, in non-exhaustive manner:

styrenics: including styrene-isoprene-styrene, styrene-butadiene-styrene, styrene-ethylene/butylene-styrene, and styrene-ethylene/propylene-styrene physically cross-linked block copolymers, for example. These products are available under the trade name Kraton® (e.g. the Kraton G and Kraton D product ranges).

By way of example, mention may also be made of styrene-butadiene-methylmethacrylate physically cross-linked block polymers sold by the supplier Arkema in the Nanostrength product range;

polyacrylics: including methylmethacrylate-butylacrylate-methylmethacrylate physically cross-linked block copolymers sold by the supplier Arkema in the Nanostrength product range, for example. By way of example, mention may also be made of acrylic/styrene UV cross-linked aqueous dispersions such as Acronal DS 6252 by BASF;

silicones: including, chemically cross-linked silicone rubber available under the trade name Elastosil by Wacker (e.g. Elastosil N2010), and acrylic/silicone copolymers, for example;

polycondensates (including polyurethanes, acrylic polyurethanes, silicone polyurethanes, polyureas, polyurea polyurethanes, polyester polyurethanes, polyether polyurethanes, polyesters, polyamides, polyester-amides, epoxy compounds) such as polydimethylsiloxane-urea physically cross-linked copolymers from the Geniomer range sold by the supplier Wacker, for example; and

mixtures thereof.

In other preferred exemplary embodiments of the present invention, the film-forming polymer has at least one Tg that is greater than ambient temperature and a molecular weight that is greater than the critical entanglement weight of the polymer. Examples of polymers that are suitable for the invention include, in non-exhaustive manner:

homo- and co-polymers of esters of (meth)acrylic acid and (meth)acrylic amide, in particular polymers resulting from the polymerization or copolymerization of methyl, ethyl, propyl, butyl, isobutyl, tertiobutyl, pentyl, hexyl, cyclohexyl, 2 ethylhexyl, heptyl, octyl, isobornyl, norbornyl, or adamantyl acrylates and/or methacrylates, or the corresponding (meth)acrylamides. Said polymers preferably comprise up to 20% of a polar co-monomer such as, for example, (meth)acrylic acid, (meth)acrylamide, hydroxyethyl(meth)acrylate, 2-hydroxypropyl(meth)acrylate, (meth)acrylonitrile, and mixtures thereof. They may also be obtained by copolymerization of at least one of the monomers mentioned with styrene or a substituted styrene;

vinyl ester or amide homo- and co-polymers, in particular homo- and co-polymers resulting from the polymerization of vinyl acetate, vinyl propionate, or vinyl versatate, with or without the presence of a polar co-monomer such as crotonic acid, allyloxyacetic acid, maleic anhydride (or acid), itaconic anhydride (or acid), vinyl acetamide or vinyl formamide. Such polymers may also be obtained by copolymerization of at least one of the monomers mentioned with styrene or a substituted styrene. Thus, vinyl polymers that are suitable for the invention may result from the homopolymerization or copolymerization of monomers selected from vinyl esters, styrene, and butadiene. Examples of vinyl esters that may be mentioned are vinyl acetate, vinyl neodecanoate, vinyl pivalate, vinyl benzoate and vinyl t-butyl benzoate;

celluloses and cellulose derivatives, e.g. cellulose esters such as cellulose acetates, cellulose propionates, cellulose butyrates, cellulose acetopropionates and cellulose acetobutyrates;

polycondensates, preferably selected from the following polymers and copolymers: polyurethanes, acrylic polyurethanes, polyureas, polyurea polyurethanes, polyester polyurethanes, polyether polyurethanes, polyesters, polyester-amides, fatty chain polyesters, epoxy compounds; and

mixtures thereof.

The polyesters include those obtained in known manner by polycondensation of aliphatic or aromatic dibasic acids with aliphatic or aromatic diols or polyols.

Aliphatic dibasic acids that may be used are succinic acid, glutaric acid, adipic acid, pimelic acid, suberic acid or sebacic acid.

Aromatic dibasic acids that may be used are terephthalic acid and isophthalic acid, or a derivative such as phthalic anhydride.

Aliphatic diols that may be used are ethylene glycol, propylene glycol, diethylene glycol, neopentyl glycol, dimethanol cyclohexane, and 4,4′-(1-methylpropylidene)-bisphenol. Polyols that may be used are glycerol, pentaerythritol, sorbitol, and trimethylol propane.

In other particular exemplary embodiments, the aqueous dispersions of particles, the aqueous dispersions of film-forming polymers, or latex may be used in the polymeric layer.

For example, the polymeric layer may result from evaporating off the aqueous phase of an aqueous dispersion of film-forming polymer particles. Film-forming polymers that are suitable for such uses include, in non-exhaustive manner, synthetic polymers of the polycondensate or free-radical type, polymers of natural origin, and mixtures thereof. Specific examples of film-forming polymers that are suitable for the invention and that may be cited, in non-exhaustive manner, are polycondensates, polyurethanes, acrylic polyurethanes, polyurethane-polyvinylpyrrolidones, polyester polyurethanes, polyether polyurethanes, polyureas, polyurea-polyurethanes, polyesters, polyester-amides, fatty chain polyesters, polyamides, epoxy ester resins, and mixtures thereof.

Free-radical type polymers may in particular be acrylic and/or vinyl polymers or copolymers.

Preferably, the free-radical type polymers are selected from anionic free-radical type polymers.

Monomers carrying an anionic group that may be used during free-radical polymerization that may be mentioned are acrylic acid, methacrylic acid, crotonic acid, maleic anhydride, and 2-acrylamido-2-methylpropanesulfonic acid.

The acrylic polymers may result from copolymerization of monomers selected from esters and/or amides of acrylic acid or methacrylic acid. Examples of ester type monomers that may be mentioned are methyl methacrylate, ethyl methacrylate, butyl methacrylate, isobutyl methacrylate, 2-ethylhexyl methacrylate, and lauryl methacrylate. Examples of amide type monomers that may be mentioned are N-t-butylacrylamide and N-t-octylacrylamide.

It is also possible to mention polymers resulting from free-radical polymerization of one or more free-radical monomers in and/or partially on the surface of pre-existing particles of at least one polymer selected from the group comprising polyurethanes, polyureas, polyesters, polyester amides, and/or alkyds. Said polymers are generally termed “hybrid polymers”.

In more particularly preferred exemplary embodiments, the film-forming polymer is selected from cellulose derivatives, more particularly from cellulose esters, and preferably from cellulose acetopropionates and cellulose acetobutyrates.

Advantageously, the polymeric layer may comprise film-forming polymer(s) at a content lying in the range 1% to 100% by weight relative to the total weight of the polymeric layer, preferably in the range 25% to 100% by weight, and more particularly in the range 50% to 100% by weight.

Film-Modifying Agent

In preferred exemplary embodiments of the present invention, the polymeric layer may further include at least one film-modifying agent.

In the context of the present invention, the term “film-modifying agent” means an agent that is capable of modifying any physical property of the film, such as resistance to traction, elongation, brittleness, and/or flexibility, for example.

Film-modifying agents that are suitable for the invention include, in non-exhaustive manner, plasticizers, coalescence agents, tackifier agents, humectants, and preferably plasticizers.

Examples of film-modifying agents that are suitable for the invention and that may be mentioned are: glycols and their derivatives such as diethylene glycol ethyl ether, diethylene glycol methyl ether, diethylene glycol butyl ether, diethylene glycol hexyl ether, ethylene glycol ethyl ether, ethylene glycol butyl ether, and ethylene glycol hexyl ether; glycol esters; propylene glycol derivatives, in particular propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether and diethylene glycol methyl ether, propylene glycol butyl ether; acid esters, in particular carboxylic acids such as citrates, in particular triethyl citrate, tributyl citrate, triethyl acetyl citrate, tributyl acetyl citrate, 2-triethylhexyl acetyl citrate; phthalates, in particular diethyl phthalate, dibutyl phthalate, dioctyl phthalate, dipentyl phthalate, dimethoxyethyl phthalate; phosphates, in particular tricresyl phosphate, tributyl phosphate, triphenyl phosphate, tributoxyethyl phosphate; tartrates, in particular dibutyl tartrate; adipates; carbonates; sebacates; benzyl benzoate; butyl acetylricinoleate; glyceryl acetylricinoleate; butyl glycolate; camphor; glycerol triacetate; N-ethyl-o,p-toluenesulfonamide; oxyethylenated derivatives such as oxyethylenated oils, in particular vegetable oils such as castor oil; silicone oils; and mixtures thereof.

As described above, a film-modifying agent is preferably selected from plasticizers, and in particular from polyesters plasticizers, in particular those sold under the trade name Resoflex R296 by the supplier Cambridge Industries of America.

Preferably, the polymeric layer may contain film-modifying agents at a content lying in the range 0.1% to 99% by weight relative to the total weight of the polymeric layer, preferably in the range 1% to 75% by weight, and more particularly in the range 10% to 50% by weight.

Advantageously, the film-modifying agent and the film-forming agent are present in the polymeric layer in a weight ratio lying in the range 10:1 to 1:100, preferably in the range 2:1 to 1:10, and more particularly in the range 1:1 to 1:5.

In particularly preferred exemplary embodiments, and as described above, an article of the present invention may comprise at least:

an adhesive layer as defined above having a first surface and a second surface opposite from said first surface; and

a polymeric arrangement that can be constituted by one or more layers including at least one film-forming polymer and having a first surface in contact with said first surface of the adhesive layer and a second surface opposite from said first surface, the second surface of the arrangement of polymeric layers having, when the article is exempt of solvents, Persoz hardness that is less than 50 s, preferably less than 40 s, more particularly less than 35 s, and even more preferably less than 30 s, and resistance to abrasion corresponding to weight loss that is less than 50 mg, preferably less than 40 mg, more particularly less than 30 mg, and even more preferably less than 20 mg, the polymeric arrangement being configured in such a manner that, when the article is exempt of solvents, the elongation at rupture of the article is greater than 30%, preferably greater than 40%, more particularly greater than 50%, and better greater than 60%, the polymeric arrangement comprising at least:

a) a first layer having said first surface and comprising i) a film-forming polymer, and ii) a film-modifying agent such as described above; and

b) a second layer adjacent to said first layer and having said second surface of the polymeric arrangement, said second layer comprising:

(i) a film-forming polymer selected from:

1) homo- and co-polymers of esters of (meth)acrylic acid and/or (meth)acrylic amide, in particular polymers resulting from the polymerization or copolymerization of methyl, ethyl, propyl, butyl, isobutyl, tertiobutyl, pentyl, hexyl, cyclohexyl, 2-ethylhexyl, heptyl, octyl, isobornyl, norbornyl, or adamantyl acrylates and/or methacrylates, or the corresponding (meth)acrylamides.

Said polymers preferably include in the range 0 to 20% by weight of a polar co-monomer such as (meth)acrylic acid, (meth)acrylamide, hydroxyethyl(meth)acrylate, 2 hydroxypropyl(meth)acrylate, and (meth)acrylonitrile.

They may also result from copolymerization with styrene or a substituted styrene;

2) vinyl ester or amide homo- and copolymers, in particular homo- and co-polymers resulting from the polymerization of vinyl acetate, vinyl propionate, or vinyl versatate, with or without the presence of a polar co-monomer such as crotonic acid, allyloxyacetic acid, maleic anhydride (or acid), itaconic anhydride (or acid), vinyl acetamide or vinyl formamide.

They may also result from copolymerization of at least one of the monomers mentioned with styrene or a substituted styrene;

3) celluloses and cellulose derivatives, e.g. nitrocelluloses and/or cellulose esters such as cellulose acetates, cellulose propionates, cellulose butyrates, cellulose acetopropionates, and cellulose acetobutyrates;

4) polycondensates, preferably selected from the following polymers and copolymers: polyurethanes, acrylic polyurethanes, polyureas, polyurea polyurethanes, polyester polyurethanes, polyether polyurethanes, polyesters, polyester-amides, fatty chain polyesters, epoxy compounds.

The polyesters include those obtained in known manner by polycondensation of aliphatic or aromatic dibasic acids with aliphatic or aromatic diols or polyols.

Aliphatic dibasic acids that may be used are succinic acid, glutaric acid, adipic acid, pimelic acid, suberic acid or sebacic acid.

Aromatic dibasic acids that may be used are terephthalic acid and isophthalic acid, or alternatively a derivative such as phthalic anhydride.

Aliphatic diols that may be used are ethylene glycol, propylene glycol, diethylene glycol, neopentyl glycol, dimethanol cyclohexane, and 4,4′-(1-methylpropylidene)bisphenol.

Polyols that may be used are glycerol, pentaerythritol, sorbitol, and trimethylol propane.

In other exemplary embodiments of the invention, the polymeric layer comes from evaporating off the aqueous phase of an aqueous dispersion of film-forming polymer particles. The film-forming polymer may then be selected from aqueous dispersions of film-forming polymer particles, and the composition of the invention may then comprise at least one aqueous phase.

The aqueous dispersion comprising one or more film-forming polymers may be prepared by the person skilled in the art using general knowledge, in particular by emulsion polymerization or by dispersing the formed polymer.

Film-forming polymers of this type that are suitable for use in a composition of the present invention and that may be mentioned are synthetic polymers of the polycondensate or free-radical type, polymers of natural origin, and mixtures thereof. Specific examples of such materials include, in non-exhaustive manner, the above-mentioned polymers (homo- and co-polymers), and more particularly the polymers from the above-mentioned classes 1-3.

Examples of polycondensates that may be mentioned are anionic, cationic, non ionic or amphoteric polyurethanes, polyurethane-acrylics, polyurethane-polyvinylpyrrolidones, polyester-polyurethanes, polyether-polyurethanes, polyureas, polyurea polyurethanes, and mixtures thereof.

It is also possible to mention polyesters, polyester amides, fatty chain polyesters, polyamides, and epoxy ester resins. Such polyesters may be obtained in known manner by polycondensation of aliphatic or aromatic dibasic acids with aliphatic or aromatic diols or polyols.

Aliphatic dibasic acids that may be used are succinic acid, glutaric acid, adipic acid, pimelic acid, suberic acid or sebacic acid.

Aromatic dibasic acids that may be used are terephthalic acid and isophthalic acid, or alternatively a derivative such as phthalic anhydride.

Aliphatic diols that may be used are ethylene glycol, propylene glycol, diethylene glycol, neopentyl glycol, dimethanol cyclohexane, and 4,4′-(1-methylpropylidene)bisphenol.

Polyols that may be used are glycerol, pentaerythritol, sorbitol, and trimethylol propane.

Free-radical type polymers may in particular be acrylic and/or vinyl polymers or copolymers.

Preferably, the free-radical type polymers are selected from anionic free-radical type polymers.

Monomers carrying an anionic group that may be used during free-radical polymerization that may be mentioned are acrylic acid, methacrylic acid, crotonic acid, maleic anhydride, and 2-acrylamido-2-methylpropanesulfonic acid.

The acrylic polymers may result from copolymerization of monomers selected from esters and/or amides of acrylic acid or methacrylic acid. Examples of ester type monomers that may be mentioned are methyl methacrylate, ethyl methacrylate, butyl methacrylate, isobutyl methacrylate, 2-ethylhexyl methacrylate, and lauryl methacrylate. Examples of amide type monomers that may be mentioned are N-t-butylacrylamide and N-t-octylacrylamide.

The vinyl polymers may result from the homopolymerization or copolymerization of monomers selected from vinyl esters, styrene, and butadiene. Examples of vinyl esters that may be mentioned are vinyl acetate, vinyl neodecanoate, vinyl pivalate, vinyl benzoate, and vinyl t-butyl benzoate. Acrylic/silicone copolymers may also be used.

It is also possible to mention polymers resulting from free-radical polymerization of one or more free-radical monomers in and/or partially on the surface of pre-existing particles of at least one polymer selected from the group comprising polyurethanes, polyureas, polyesters, polyester amides, and/or alkyds. Said polymers are generally termed “hybrid polymers”;

(ii) a film-modifying agent; and

(iii) a coloring substance as defined below, in particular selected from pigments.

Thus, in these variant exemplary embodiments, The first polymeric layer, also referred to as a “top-coat” since it is for being in direct contact with the outside environment once the flexible article has been applied to the nail or false nail, is coated onto the second polymeric layer, also referred to as a “colored layer” since it is dedicated in particular to bringing a color and/or a desired cosmetic effect to said flexible article.

In other words, in the meaning of this variant embodiment, the second polymeric layer or colored layer is intermediate to the adhesive layer and the first polymeric layer or top-coat.

Thus, the first and second polymeric layers may differ, firstly by the content and/or the nature of the various compounds characterizing them, and secondly by the presence, in particular in the second polymeric layer, of any additive commonly used in the field of the present invention, in particular coloring substances as defined below.

Other Additives

i) Coloring Substances

The adhesive layer(s) and/or the polymeric layer(s) of the invention, preferably the polymeric layer(s), may further include at least one organic or inorganic coloring substance, in particular of pigment or nacre type conventionally used in cosmetic compositions.

The quantities of these various ingredients are those conventionally used in this field, for example in the range 0.01% to 20% by weight relative to the total weight of the adhesive and/or polymeric layers, and in particular in the range 0.01% to 10% by weight.

The term “pigments” means white or colored mineral or organic particles that are insoluble in the aqueous medium that is to color and/or opacify the resulting film.

Mineral pigments that are suitable for use in the invention and that may be mentioned are oxides of titanium, zirconium, or cerium and oxides of zinc, iron, or chromium, ferric blue, manganese violet, ultramarine blue, and chromium hydrate.

The pigment may also be a pigment having a structure that may for example by of sericite or brown iron oxide or titanium dioxide or silica type. By way of example, such a pigment is sold under the reference NS or NJ Coverleaf by Chemicals and Catalysts and presents a contrast ratio close to 30.

The coloring substance may also comprise a pigment having a structure that may for example be of silica-microsphere type containing iron oxide. An example of a pigment presenting such a structure is the pigment sold by Miyoshi under the reference PC Ball PC-LL-100 P, the pigment being constituted by silica microspheres containing yellow iron oxide.

Amongst organic pigments usable in the invention, mention may be made of: carbon black; D & C type pigments; lakes based on cochenille carmine, on barium, strontium, calcium, or aluminum; or even diceto pyrrolopyrroles (DPPs) described in EP-A-0 542 669, EP-A-0 787 730, EP-A-0 787 731, and WO-A-96/08537.

The term “nacres” means colored particles of any form, which may optionally be iridescent, as produced in the shells of certain mollusks, or which are synthesized, and that exhibit a “pearlescent” coloring effect by optical interference.

Nacres may be selected from nacre pigments such as titanium mica coated with iron oxide, mica coated with bismuth oxychloride, titanium mica coated with chromium oxide, titanium mica coated with an organic colorant, and nacre pigments based on bismuth oxychloride. They may also be particles of mica on the surface of which at least two successive layers of metal oxides and/or organic coloring materials have been superposed.

As examples of nacres, mention may also be made of natural mica coated with titanium oxide, iron oxide, natural pigment, or bismuth oxychloride.

Commercially available nacres that may be mentioned are TIMICA, FLAMENCO and DUOCHROME (mica based) sold by the supplier ENGELHARD, TIMIRON nacres sold by the supplier MERCK, PRESTIGE mica based nacres sold by the supplier ECKART, and SUNSHINE synthetic mica based nacres sold by the supplier SUN CHEMICAL.

More particularly, the nacres may have a yellow, pink, red, bronze, orangey, brown, gold, and/or coppery color or glint.

Illustrative examples of nacres suitable for being implemented in the context of the present invention that may be mentioned are gold color nacres, in particular those sold by ENGELHARD under the trade names Brillant gold 212G (Timica), Gold 222C (Cloisonne), Sparkle gold (Timica), Gold 4504 (Chromalite), and Monarch gold 233X (Cloisonne); bronze nacres, in particular those sold by MERCK under the trade names Bronze fine (17384) (Colorona) and Bronze (17353) (Colorona), and by ENGELHARD under the trade name Super bronze (Cloisonne); orange nacres especially those sold by ENGELHARD under the trade names Orange 363C (Cloisonne) and Orange MCR 101 (Cosmica), and by MERCK under the trade names Passion orange (Colorona) and Matte orange (17449) (Microna); brown-tinted nacres sold by ENGELHARD under the trade names Nu-antique copper 340XB (Cloisonne) and Brown CL4509 (Chromalite); nacres with a copper glint sold by ENGELHARD under the trade name Copper 340A (Timica); nacres with a red glint, especially those sold by MERCK under the trade name Sienna fine (17386) (Colorona); nacres with a yellow glint, especially those sold by ENGELHARD under the trade name Yellow (4502) (Chromalite); red-tinted nacres with gold glints, especially those sold by ENGELHARD under the trade name Sunstone G012 (Gemtone); pink nacres, especially those sold by ENGELHARD under the trade name Tan opale G005 (Gemtone); black nacres with a glint, especially those sold by ENGELHARD under the trade name Nu antique bronze 240 AB (Timica); blue nacres, especially those sold by MERCK under the trade name Matte blue (17433) (Microna); white nacres with silvery glints, especially those sold by MERCK under the trade name Xirona Silver; and orange-pink green-gold highlight nacres sold by MERCK under the trade names Indian summer (Xirona); and mixtures thereof.

Examples of liposoluble colorants are Sudan red, D&C Red No.17, D&C Green No.6, β-carotene, soybean oil, Sudan brown, D&C Yellow No.11, D&C Violet No.2, D&C orange No.5, and quinoline yellow. Examples of hydrosoluble colorants are beetroot juice and methylene blue.

ii) Effect Material

The adhesive layer and/or the polymeric layer of the invention may further include at least one material having a specific optical effect.

This effect differs from a simple conventional hue effect, i.e. uniform and stabilized such as that produced by conventional coloring materials described above, such as monochromatic pigments for example.

In the meaning of the invention, “stabilized” signifies a lack of variability in color with the angle of observation, or even in response to a change in temperature.

Said material is present in a quantity sufficient to produce an optical effect that is visible to the naked eye. Advantageously, it is an effect selected from goniochromatic, metallic especially mirror, soft-focus, rainbow, thermochromic, and/or photochromic effects.

As an example, said material may be selected from particles with a metallic glint, goniochromatic coloring agents, diffractive pigments, thermochromic agents, photochromic agents, optical whitening agents, as well as fibers, in particular interferential fibers. Clearly, said various materials may be combined to simultaneously produce two effects or even a novel effect within the context of the invention.

Particles with a Metallic Glint

The term “particles with a metallic glint” denotes particles of nature, size, structure, and surface condition that allows them to reflect incident light, in particular in a non iridescent manner.

Particles with a substantially planar outer surface are also suitable since they can readily produce intense specular reflection that may be qualified as a mirror effect if their size, structure and surface condition allow.

The particles with a metallic glint of the invention may, for example, reflect light in all of its visible components without significantly absorbing one or more wavelengths. The spectral reflectance of said particles may, for example, be more than 70% in the 400 nanometers (nm) to 700 nm range, preferably at least 80% or even 90% or 95%.

Said particles are generally 1 μm or less in thickness, in particular 0.7 μm or less in thickness, and in particular 0.5 μm or less in thickness.

In particular, the particles with a metallic glint that are used in accordance with the invention are selected from:

particles of at least one metal and/or at least one metallic derivative;

particles comprising a substrate, that may be organic or mineral, a mono-material or a multi-material, at least partially covered with at least one layer with a metallic glint comprising at least one metal and/or at least one metallic derivative; and

mixtures of said particles.

Examples that may be mentioned of metals that may be present in said particles are Ag, Au, Cu, Al, Ni, Sn, Mg, Cr, Mo, Ti, Zr, Pt, Va, Rb, W, Zn, Ge, Te, Se and mixtures or alloys thereof. Ag, Au, Cu, Al, Zn, Ni, Mo, Cr and mixtures or alloys thereof (for example bronzes and brasses) are preferred metals.

The term “metallic derivatives” means compounds derived from metals, in particular oxides, fluorides, chlorides, and sulfides.

Examples that may be mentioned of metallic derivatives that may be present in said particles are metallic oxides such as oxides of titanium, in particular TiO₂, of iron, in particular Fe₂O₃, of tin, of chromium, barium sulfate and the following compounds: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂, and mixtures or alloys thereof.

In a first variation, the particles with a metallic glint may be composed of at least one metal as defined above, at least one metallic derivative as defined above or a mixture thereof.

Said particles may be at least partially covered with a layer of another material, for example a transparent material such as rosin, silica, stearates, polysiloxanes, polyester resins, epoxy resins, polyurethane resins, or acrylic resins.

Illustrative examples of such particles that may be mentioned are particles of aluminum, such as those sold under the trade names STARBRITE 1200 EAC® by SIBERLINE and METALURE® by ECKART.

It is also possible to mention copper powders or alloy mixtures such as reference 2844 sold by RADIUM BRONZE, metallic pigments such as aluminum or bronze, such as those sold under the trade name ROTOSAFE 700 by ECKART, particles of aluminum coated with silica sold under the trade name VISIONAIRE BRIGHT SILVER by ECKART and particles of metal alloy such as bronze powders (copper and zinc alloy) coated with silica sold as VISIONAIRE BRIGHT NATURAL GOLD by ECKART.

In a second variation, said particles may be particles comprising a substrate and that thus present a structure that is multilayered, for example two-layered. Said substrate may be organic or inorganic, natural or synthetic, a mono- or multi-material, solid or hollow. When the substrate is synthetic, it may be produced in a form encouraging the formation of a reflective surface after coating, in particular after depositing a layer of material with a metallic glint. The substrate may, for example, have a planar surface and the layer of materials with a metallic glint may have substantially uniform thickness.

In particular, the substrate may be selected from the metals and metallic derivatives mentioned above, and also from glasses, ceramics, aluminas, silicas, silicates and in particular aluminosilicates and borosilicates, synthetic mica such as fluorophlogopite, and mixtures thereof, this list not being limiting.

The layer with a metallic effect may completely or partially coat the substrate and said layer may be at least partially coated with a layer of another material, for example a transparent material as mentioned above. In a particular implementation, said layer with a metallic glint completely coats the substrate directly or indirectly, i.e. with the interposition of at least one metallic or non metallic intermediate layer.

The metals or metallic derivatives that may be used in the reflective layer are as defined above. As an example, it may be formed by at least one metal selected from silver, aluminum, chromium, nickel, molybdenum, gold, copper, tin, magnesium and mixtures (alloys) thereof. More particularly, sliver, chromium, nickel, molybdenum, and mixtures thereof may be used.

An illustrative example of said second type of particle that may be mentioned is as follows:

particles of glass coated with a metallic layer, in particular those described in JP-A-09188830, JP-A-10158450, JP-A-10158541, JP-A-07258460, and JP-A-05017710.

Illustrative examples of such particles comprising a glass substrate that may be mentioned are those coated respectively with silver, gold or titanium in the form of flakes sold by NIPPON SHEET GLASS under the MICROGLASS METASHINE trade names. Particles with a glass substrate coated with silver in the form of flakes are sold under the trade name MICROGLASS METASHINE REFSX 2025 PS by TOYAL. Particles with a glass substrate coated with nickel/chromium/molybdenum alloy are sold under the trade name CRYSTAL STAR GF 550, GF 2525 by the same firm. Those coated with either brown iron oxide or titanium oxide, tin oxide, or a mixture thereof, such as those sold under the trade name REFLECKS by ENGELHARD or those sold under the trade name METASHINE MC 2080GP by NIPPON SHEET GLASS are also known.

Said glass particles coated with metals may be coated with silica, such as those sold under the trade name METASHINE series PSS1 or GPS1 by NIPPON SHEET GLASS.

Particles with a spherical glass substrate that may or may not be coated with a metal, in particular those sold under the trade name PRIZMALITE MICROSPHERE by PRIZMALITE INDUSTRIES.

Pigments from the METASHINE 1080R range sold by NIPPON SHEET GLASS Co. LTD are also suitable. Said pigments, more particularly those described in Japanese patent JP 2001 11340, are C-GLASS glass flakes comprising 65% to 72% of SiO2 coated with a layer of rutile (TiO₂) type titanium oxide. Said glass flakes have a mean thickness of 1 μm and a mean size of 80 μm, giving a mean size/mean thickness ratio of 80. They have blue, green, yellow or silver glints depending on the thickness of the TiO₂ layer.

Particles comprising a borosilicate substrate coated with silver, also known as “white nacres”.

Particles with a metallic substrate such as aluminum, copper, bronze, in the form of flakes, are sold under the trade name STARBRITE by SILBERLINE and under the trade name VISIONAIR by ECKART.

Particles comprising a synthetic mica substrate coated with titanium dioxide, for example particles with a dimension in the range 80 μm to 100 μm comprising a synthetic mica (fluorophlogopite) substrate coated with titanium dioxide representing 12% of the total weight of the particle are sold under the trade name PROMINENCE by NIHON KOKEN.

The particles with a metallic glint may also be selected from particles formed by a stack of at least two layers with different refractive indices. Said layers may be polymeric or metallic in nature and in particular may include at least one polymeric layer.

Thus, the particles with a metallic effect may be particles deriving from a multilayer polymeric film.

The choice of materials intended to constitute the various layers of the multilayer structure is clearly made so as to provide the particles formed with the desired metallic effect.

Such particles have in particular been described in International patent application WO-A-99/36477 and U.S. Pat. No. 6,299,979 and U.S. Pat. No. 6,387,498 and are more particularly identified in the section on goniochromatic effects.

Diffractive Pigments

The term “diffractive pigment” as used in the meaning of the present invention designates a pigment that is capable of producing a variation in color depending on the angle of observation when lit by white light, because of the presence of a structure that diffracts the light.

A diffractive pigment may include a diffraction grating that is capable of diffracting an incident ray of monochromatic light in defined directions.

The diffraction grating may comprise a periodic pattern, in particular a line, with the distance between two adjacent patterns being the same as the wavelength of the incident light.

When the incident light is polychromatic, the diffraction grating separates the various spectral components of the light and produces a rainbow effect.

With regard to the structure of diffractive pigments, reference can usefully be made to the article “Pigments Exhibiting Diffractive Effects” by Alberto Argoitia and Matt Witzman, 2002, Society of Vacuum coaters, 45th Annual Technical Conference Proceedings 2002.

The diffractive pigment may be made with patterns having various profiles, in particular triangular, optionally symmetrical, notched, of optionally constant width, or sinusoidal.

The spatial frequency of the grating and the depth of the patterns are selected as a function of the degree of separation of the various desired orders. The frequency may be in the range 500 lines per mm to 3000 lines per mm, for example.

Each of the particles of the diffractive pigment preferably presents a flat shape, and in particular a wafer shape.

A single pigment particle may include two crossed diffraction gratings that are optionally perpendicular.

A possible structure for the diffractive pigment may comprise a layer of reflective material that is covered on at least one side by a layer of dielectric material. The dielectric material may make the diffractive pigment stiffer and longer lasting. The dielectric material may thus, for example, be selected from the following materials: MgF₂, SiO₂, Al₂O₃, AlF₃, CeF₃, LaF₃, NdF₃, SmF₂, BaF₂, CaF₂, LiF, and combinations thereof. The reflective material may, for example, be selected from metals and their alloys and also from non metallic reflective materials. Metals that may be used and that may be mentioned are Al, Ag, Cu, Au, Pt, Sn, Ti, Pd, Ni, Co, Rd, Nb, Cr and compounds, combinations and alloys thereof. Such a reflective material may alone constitute the diffractive pigment that will then be a monolayer.

In a variant, the diffractive pigment may include a multilayer structure comprising a core of dielectric material with a reflective layer covering at least one side, or indeed completely encapsulating, the core. A layer of dielectric material may also cover the reflective layer(s). The dielectric material used is thus preferably inorganic, and may, for example, be selected from metal fluorides, metal oxides, metal sulfides, metal nitrides, metal carbides, and combinations thereof The dielectric material may be in the crystalline, semi-crystalline, or amorphous state. In this configuration, the dielectric material may, for example, be selected from the following materials: MgF₂, SiO, SiO₂, Al₂O₃, TiO₂, WO, AlN, BN, B₄C, WC, TiC, TiN, N₄Si₃, ZnS, glass particles, diamond type carbon and combinations thereof.

The diffractive pigment used may in particular be selected from those described in US 2003/0031870.

A diffractive pigment may, for example, have the following structure: MgF₂/Al/MgF₂; a diffractive pigment with that structure is sold under the trade name SPECTRAFLAIR 1400 Pigment Silver by FLEX PRODUCTS, or SPECTRAFLAIR 1400 Pigment Silver FG. The proportion by weight of MgF₂ may lie in the range 80% to 95% of the total weight of the pigment.

Goniochromatic Coloring Agents

In the context of the invention, a goniochromatic coloring agent allows a color change, also termed a “color flop”, to be observed as a function of the angle of observation, that is higher than that which may be obtained with nacres. One or more goniochromatic coloring agents may be used simultaneously.

The goniochromatic coloring agent may be selected to present a relatively large color change with the angle of observation.

The goniochromatic coloring agent may thus be selected so that for a variation in the angle of observation in the range 0 to 80°, illuminated at 45°, a variation ΔE of at least 2 is observed in the color of the cosmetic composition, measured in the CIE 1976 colorimetric space.

The goniochromatic coloring agent may also be selected so that for an illumination at 45° and a variation in the angle of observation in the range 0 to 80°, a variation Dh of at least 30° or even at least 40° or at least 60° or even at least 100° is observed in the angle of the hue of the cosmetic composition, using the CIE 1976 colorimetric space.

By way of example, the goniochromatic coloring agent may be selected from multilayer interference structures and liquid crystal coloring agents.

By way of example, a multilayer structure may comprise at least two layers, each layer, independently or otherwise of the other layer(s), being produced, for example, from at least one material selected from the group constituted by the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers, and combinations thereof.

The multilayer structure may optionally be symmetrical with respect to a central layer as regards the chemical nature of the stacked layers.

Examples of symmetrical multilayer interference structures that may be used in compositions produced in accordance with the invention are as follows: Al/SiO₂/Al/SiO₂/Al, pigments with this structure being sold by DUPONT DE NEMOURS; Cr/MgF₂/Al/MgF₂/Cr, pigments with this structure being sold under the trade name CHROMAFLAIR by FLEX; MoS₂/SiO₂/Al/SiO₂/MoS₂; Fe₂O₃/SiO₂/Al/SiO₂/Fe₂O₃, and Fe₂O₃/SiO₂/Fe₂O₃/SiO₂/Fe₂O₃/Fe₂O₃, pigments having these structures being sold under the trade name SICOPEARL by BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the trade name XIRONA by MERCK (Darmstadt). By way of example, said pigments may be pigments with a silica/titanium oxide/tin oxide structure sold under the trade name XIRONA MAGIC by MERCK, pigments with a silica/ brown iron oxide structure sold under the trade name XIRONA INDIAN SUMMER by MERCK and pigments with a silica/titanium oxide/mica/tin oxide structure sold under the trade name XIRONA CARRIBEAN BLUE by MERCK. INFINITE COLORS pigments by SHISEIDO may also be mentioned. Depending on the nature of the various layers, different effects are obtained. Thus, with the Fe₂O₃/SiO₂/Al/SiO₂/Fe₂O₃ structure, the color passes from golden green to red-gray for SiO₂ layers from 320 nm to 350 nm; from red to golden for SiO₂ layers from 380 nm to 400 nm; from violet to green for SiO₂ layers from 410 nm to 420 nm; from copper to red for SiO₂ layers from 430 nm to 440 nm.

It is also possible to use goniochromatic coloring agents with a multilayer structure comprising alternating polymeric layers.

A non-limiting list of illustrative materials that may constitute the various layers of the multilayer structure that may be mentioned is as follows: polyethylene naphthalate (PEN) and its isomers, for example 2,6-, 1,4-, 1,5-, 2,7- and 2,3-PEN, polyalkylene terephthalates, polyimides, polyetherimides, atactic polystyrenes, polycarbonates, polymethacrylates and alkyl polyacrylates, syndiotactic polystyrene (sPS), syndiotactic poly-alpha-methylstyrenes, syndiotactic polydichlorostyrene, copolymers and mixtures of said polystyrenes, cellulose derivatives, polyalkylene polymers, fluorinated polymers, chlorinated polymers, polysulfones, polyethersulfones, polyacrylonitriles, polyamides, silicone resins, epoxy resins, polyvinyl acetate, polyether-amides, ionomeric resins, elastomers and polyurethanes. Copolymers are also suitable, for example copolymers of PEN (for example, copolymers of 2,6-, 1,4-, 1,5-, 2,7-, and/or 2,3-naphthalene dicarboxylic acid or its esters with (a) acid terephthalic or its esters; (b) isophthalic acid or its esters; (c) phthalic acid or its esters; (d) alkane glycols; (e) cycloalkane glycols (for example cyclohexane dimethanol diol); (f) alkane dicarboxylic acids; and/or (g) cycloalkane dicarboxylic acids, copolymers of polyalkylene terephthalates and styrene copolymers. Furthermore, each individual layer may include mixtures of two or more of the above polymers or copolymers. The choice of materials intended to constitute the various layers of the multilayer structure is clearly made so as to endow the particles formed with the desired optical effect.

Examples of pigments with a polymeric multilayer structure that may be mentioned are those sold by 3M under the trade name COLOR GLITTER.

By way of example, liquid crystal coloring agents comprise silicones, or cellulose ethers onto which mesomorphic groups have been grafted.

Examples of suitable liquid crystal goniochromatic particles are those sold by CHENIX, and those sold under the trade name HELICONE® HC by WACKER.

Said agents may also be in the form of dispersed goniochromatic fibers. Said fibers may, for example, be of a size in the range 50 μm to 700 μm, for example about 300 μm. In particular, it is possible to use interference fibers with a multilayer structure. Fibers with a multilayer polymer structure have in particular been described in EP A 0 921 217 and EP A 0 686 858 and in U.S. Pat. No. 5,472,798. The multilayer structure may comprise at least two layers, each layer, independently or not of the other layer(s), being produced from at least one synthesized polymer. The polymers present in the fibers may have a refractive index lying in the range 1.30 to 1.82 and preferably in the range 1.35 to 1.75. Preferred polymers for constituting the fibers are polyesters such as polyethylene terephthalate, polyethylene naphthalate, polycarbonate; acrylic polymers such as polymethyl methacrylate; polyamides.

Goniochromatic fibers with a two layered polyethylene terephthalate/nylon-6 structure are sold under the trade name TEIJIN under the trade name MORPHOTEX.

In a variation, said goniochromatic coloring agent may be associated with at least one diffractive pigment.

The combination of these two materials results in a composition or a film with an enhanced color variability, and thus that is capable of allowing an observer to perceive a color change or even color movement under a number of observation and illumination conditions.

The weight ratio of the diffractive pigment relative to the goniochromatic coloring agent is preferably in the range 85/15 to 15/85, more preferably in the range 80/20 to 20/80, still more preferably in the range 60/40 to 40/60, for example of the order of 50/50. Such a ratio is favorable to the production of a sustained rainbow effect and goniochromatic effect.

Optical Whitening Agents

Optical whitening agents are compounds that are well known to the skilled person. Such compounds have been described in “Fluorescent whitening agents, Encyclopedia of Chemical Technology, Kirk-Othmer”, vol 11, pp. 227-241, 4eme edition, 1994, Wiley. More particularly, they can be defined as compounds that absorb essentially in the UVA region between 300 nm and 390 nm and emit essentially between 400 nm and 525 nm. Optical whitening agents that may in particular be mentioned include stilbene derivatives, in particular polystyrylstilbenes and triazinstilbenes, coumarin derivatives, in particular hydroxycoumarins and aminocoumarins, oxazole, benzoxazole, imidazole, triazole, pyrazoline derivatives, pyrene derivatives and porphyrin derivatives and mixtures thereof. Said compounds are readily available commercially.

Examples that may be mentioned are as follows: the stilbene derivative of naphtho-triazole sold under the trade name “Tinopal GS”, 4,4′-di-styryl-biphenyl sulfonate di-sodium salt (CTFA name: disodium distyrylbiphenyl disulfonate) sold under the trade name “Tinopal CBS-X”, the cationic derivative of aminocoumarin sold under the trade name “Tinopal SWN CONC.”, sodium 4,4′-bis[(4,6-dianilino-1,3,5-triazin-2-yl)amino]stilbene-2,2′-disulfonate sold under the trade name “Tinopal SOP”, 4,4′-bis-[(4-anilino-6-bis(2-dihydroxyethyl)amino-1,3,5-triazin-2-yl)amino]stilbene-2,2′-disulfonic acid sold under the trade name “Tinopal UNPA-GX”, 4,4′-bis-[anilino-6-morpholine-1,3,5-triazin-2-yl)amino]stilbene sold under the trade name “Tinopal AMS-GX”, 4,4′-bis-[(4-anilino-6-(2-hydroxyethyl)methyl amino-1,3,5-triazin-2-yl)amino]stilbene-2,2′-disodium sulfonate sold under the trade name “Tinopal 5BM-GX”, all from CIBA Specialites Chimiques; 2,5-thiophene di-yl bis(5-ter-butyl-1,3-benzoxazole) sold under the trade name “Uvitex OB” by CIBA; the anionic derivative of di-aminostilbene in dispersion in water, sold under the trade name “Leucophor BSB Liquide” by CLARIANT; optical whitening lakes sold under the trade name “COVAZUR” by WACKHERR.

The optical whitening agents that may be used in the present invention may also be in the form of copolymers, for example of acrylates and/or methacrylates, grafted with optical whitening agent groups as described in FR 99/10942.

They may be used as is or introduced into the film in the form of particles and/or fibers coated with said whitening agents, such as those described below.

In particular, it is possible to use fibers coated with optical whitening agents as sold by LCW under the trade name Fiberlon 54 ZO3, with a length of about 0.4 mm and a weight of 0.5 deniers.

Material with Relief Effect

The relief effect may or may not be associated with an optical effect. A material of this type is generally present in a quantity sufficient to produce a relief effect that is perceptible to the touch or even to the naked eye. It may also be a rough and/or hammered effect.

Material Producing a Rough Effect

Particles with a substantially spherical or ovoid shape may produce a soft cosmetic touch effect. Advantageously, the solid particles have a substantially spherical shape to allow them to be distributed properly during application.

The solid particles used in accordance with the invention may have a mean size of 2.5 μm to 5 mm, preferably 50 μm to 2 mm. The smaller the particles, the better the staying power of the particles. Using particles is also compatible with producing motifs.

The solid particles may be formed from any material satisfying the density properties defined above. As an example, the solid particles may be formed from a material selected from glass, zirconium oxide, tungsten carbide, plastics such as polyurethanes, polyamides, polytetrafluoroethylene, polypropylene, metals such as steel, copper, brass, chromium, marble, onyx, jade, natural nacre, precious stones (diamond, emerald, ruby, sapphire), amethyst, aquamarine. Preferably, glass beads are used, such as those sold under the trade name “SILIBEADS®” by SIGMUND LINDNER; said beads have the further advantage of also producing a glossy and scintillating cosmetic effect.

The solid particles, that may or may not be deformable, may be solid or hollow, colorless or colored, coated or otherwise.

Regarding the fibers used in the invention, they may be fibers of synthetic or natural, mineral or organic origin.

The term “fiber” means an object with a length L and a diameter D such that L is much greater than D, D being the diameter of the circle in which the section of the fiber is inscribed. In particular, the ratio L/D (or form factor) is selected so as to be in the range 3.5 to 2500, preferably in the range 5 to 500, more preferably in the range 5 to 150.

They may be fibers used in the fabrication of textiles, in particular fibers of silk, cotton, wool, linen, cellulose fibers, especially those derived from wood, vegetable or algae, rayon, polyamide (Nylon®), viscose, acetate, in particular rayon acetate, poly-(p-phenylene-terephthalamide) (or aramide) especially Kevlar®, acrylic polymer, especially polymethyl methacrylate or poly 2-hydroxyethyl methacrylate, polyolefin and especially polyethylene or polypropylene, glass, silica, carbon in particular in the form of graphite, polytetrafluoroethylene (such as Teflon®), insoluble collagen, polyesters, polyvinyl or polyvinylidene chloride, polyvinyl alcohol, polyacrylonitrile, chitosan, polyurethane, polyethylene phthalate and fibers formed by a mixture of polymers such as those mentioned above, such as polyamide/polyester fibers.

Material Producing a Hammered Effect

The inventors have also established that, in the invention, it is possible for a material to comprise a pyrogenated silica mixture, a metallic pigment, and an organopolysiloxane compound to produce therein a hammered appearance.

Such a mixture has been described in EP A 1 040 813.

iii) Material with an olfactory effect

Advantageously, the adhesive and/or polymeric layer(s) of an article of the invention may also be provided with olfactory properties, especially by incorporating into at least one of the layers, at least one odorifying material or a fragrancing substance.

The fragrancing substance may be selected from any odorifying substance that is well known to the skilled person, in particular from essential oils and/or essences.

Said material olfactory may, if appropriate, be introduced via a solvent-plasticizer.

The term “solvent-plasticizer” means a compound that at least partially dissolves the olfactory material and that is capable of evaporating off slowly.

The solvent-plasticizer may be selected from glycols such as dipropylene glycol, ethyldiglycol, n-propylglycol, n-butylglycol, methyldiglycol, n-butyldiglycol, alcohols such as cyclohexanol, 2-ethyl butanol, 3-methoxy butanol, 2-ethyl hexanol, phenoxyethanol, esters such as glycol monoacetate, ethylglycol acetate, n-butylglycol acetate, ethyldiglycol acetate, n-butyldiglycol acetate, methyl abietate, isopropyl myristate, propylene glycol diacetate, the acetate of propylene glycol methyl ether, glycol ethers such as dipropylene glycol -methyl ether or -butyl ether, used alone or as a mixture.

Any one of the above-mentioned layers of the article of the invention may also contain one or more additives with a formulation currently used in cosmetics, and more especially in the field of cosmetics and/or nail care. They may also be selected from vitamins, oligo-elements, softeners, sequestrating agents, alkalinizing agents or acidifying agents, wetting agents, thickening agents, dispersing agents, anti-foaming agents, spreading agents, co-resins, preservatives, UV filters, active ingredients, moisturizing agents, neutralizing agents, stabilizing agents, antioxidants and mixtures thereof.

Thus, they may in particular incorporate, as active ingredients, hardening or reinforcing agents for keratinous materials, active ingredients promoting nail growth such as methylsulfonylmethane, and/or active ingredients for treating various diseases affecting the nails, such as antimycotic or antimicrobial agents, for example.

The invention is illustrated in detail in the following example that is presented by way of non-limiting illustration of the invention.

Unless indicated to the contrary, quantities are given in percentages by weight of the first material relative to the total weight of the composition.

### EXAMPLE

Making Flexible Articles of the Invention

In this example, the polymeric layer was in the form of a combination or an arrangement. It was made from two compositions of which one, referred to as the “colored layer” was dedicated to being coated with a second composition, referred to as the “top-coat”.

Composition of the “Colored Layer”

| Compounds | % |
|---|---|
| Cellulose acetopropionate⁽¹⁾ | 13.5 |
| Polyester plasticizer⁽²⁾ | 1.5 |
| Ethyl acetate⁽³⁾ | 85 |

⁽¹⁾CAP 482-20, sold by the supplier Eastman Chemical Company
⁽²⁾Resoflex R296, sold by the supplier Cambridge Industries of America
⁽³⁾Sold by the supplier Eastman Chemical

Composition of the “Top-Coat”

| Compounds | % |
|---|---|
| Cellulose acetobutyrate⁽⁴⁾ | 17.3 |
| Polyester plasticizer⁽²⁾ | 11.4 |
| Ethyl acetate⁽³⁾ | 68.2 |
| Pigments Red 7 Lake⁽⁵⁾ | 3.1 |

⁽⁴⁾CAB 381-2, sold by the supplier Eastman Chemical Company
⁽⁵⁾SUNCROMA D&C RED 7 CA LAKE C-19-003, sold by the supplier SUN

Protocol for Preparing the Two Above-Mentioned Compositions:

The ingredients, excluding pigments, were placed in a container, then mixed by means of an agitator of the Rayneri type until homogeneous mixtures were obtained. The pigments were then incorporated and mixed under agitation. The mixture obtained was ground by means of a bead grinder of the Dynomill type so as to disperse the pigments as well as possible.

A third composition, dedicated to forming the adhesive layer, was coated at 300 μm while wet on an adhesive backing (Scotchpak 1022 Release liner 3.0 mil available from the supplier 3M).

The third composition could in particular be any one of the four examples described in the table below.

| | Composition | | | |
|---|---|---|---|---|
| Compounds | 1 | 2 | 3 | 4 |
| Tackifier resin | 60% | 60% | 60% | 60% |
| REGALITE R1100 by EASTMAN | | | | |
| CHEMICAL | | | | |
| Mixture of styrene- | 15% | — | 20% | — |
| ethylene/butylene-styrene | | | | |
| linear triblock copolymers | | | | |
| and of styrene- | | | | |
| ethylene/butylene diblock | | | | |
| copolymers | | | | |
| KRATON G1657M (SEBS/SEB) by | | | | |
| KRATON POLYMERS | | | | |
| Mixture of styrene- | — | 15% | — | 20% |
| isoprene-styrene linear | | | | |
| triblock copolymers and of | | | | |
| styrene-isoprene diblock | | | | |
| copolymers | | | | |
| KRATON D1161P (SIS/SI) by | | | | |
| KRATON POLYMERS | | | | |
| Plasticizer | 25% | 25% | 20% | 20% |
| PARLEAM by NOF CORPORATION | | | | |

Protocol for Preparing the Adhesives:

The tackifier resin (Regalite R1100) and the plasticizer (Parleam) were mixed together, then heated under agitation at 170° C.

Once the mixture was liquid and homogeneous, the block copolymer mixture was incorporated progressively maintaining agitation and the temperature at 170° C. until a homogeneous mixture was obtained.

The resulting adhesive was then coated onto the face of the colored layer of the above-described polymeric film in the following ways. The laminates were then left to cool at ambient temperature.

| | Thickness of | Thickness of the | Thickness of |
|---|---|---|---|
| Composition of | the adhesive | colored layer | the top-coat |
| the adhesive | layer | after drying | after drying |
| layer | (in μm) | (in μm) | (in μm) |
| Composition 1 | 50 | 65 | 5 |
| Composition 2 | 50 | 65 | 5 |
| Composition 3 | 80 | 45 | 5 |
| Composition 4 | 80 | 45 | 5 |

After the various layers had dried, a silicone adhesive-backing film was applied to the adhesive layer. The laminate was then cut to the shape of nails so that it could then be applied to the nails. During application, care was taken beforehand to remove the adhesive backing film.

The arrangement of polymeric layers and the adhesive layers described above in examples 1 to 4 made it possible to obtain flexible articles, represented in the above table, that presented improved staying power on the nails.

1. A flexible article for application to the nails and/or false nails so as to make them up and/or care for them, the article comprising at least one adhesive layer, and being characterized in that said adhesive layer comprises at least:
two block copolymers;
a tackifier resin having a number-average molecular weight that is less than or equal to 10000 g/mol selected from rosin, rosin derivatives, hydrocarbon resins, and mixtures thereof; and
a plasticizer.

2. The article according to claim 1, wherein the block copolymers result from the polymerization of at least one olefin and at least one styrene block.

3. The article according to claim 2, wherein said at least one olefin comprises ethylene carbide monomers.

4. The article according to claim 1, wherein the block copolymers are selected from sequenced copolymers including at least one styrene block and at least one block including at least one pattern selected from butadiene, ethylene, propylene, butylene, isoprene, or a mixture thereof.

5. The article according to claim 1, wherein one of the two block copolymers is selected from a diblock copolymer and the other is selected from a triblock-, multiblock-, radial-, or star-type copolymer.

6. The article according to claim 5, wherein the weight ratio “triblock, multiblock, radial, or star copolymer”/“diblock copolymer” is less than 7.

7. The article according to claim 1, wherein the block copolymers are present at a content lying in the range 1% to 70% by weight relative to the total weight of said adhesive layer.

8. The article according to claim 1, wherein rosin derivatives are selected from rosin esters and hydrocarbon resins are selected from hydrogenated resins.

9. The article according to claim 1, wherein the adhesive layer contains tackifier resin(s) at a content lying in the range 10% to 95% by weight relative to the total weight of said adhesive layer.

10. The article according to claim 1, wherein the plasticizer is selected from hydrocarbon oils.

11. The article according to claim 1, wherein the adhesive layer contains plasticizers at a content lying in the range 5% to 40% by weight relative to the total weight of said adhesive layer of said article.

12. The article according to claim 1, wherein said article further includes at least one polymeric layer.

13. The article according to claim 12, wherein the polymeric layer includes at least one film-forming polymer.

14. The article according to claim 12, wherein the polymeric layer further comprises at least one film-modifying agent and/or at least one film-forming co-agent.

15. The article according to claim 12, wherein the polymeric layer further comprises at least one coloring substance.

16. The article according to claim 1, wherein the adhesive layer of said article presents a thickness lying in the range 30 μm to 100 μm.

17. The article according to claim 1, wherein said article presents thickness lying in the range 6 μm to 1 mm.

18. The article according to claim 1, wherein said article is capable of being removed by means of a solvent selected from acetone, alkyl acetates, and mixtures thereof.

19. The article according to claim 1, wherein the dry extract weight of said article is greater than or equal to 95% by weight relative to the total weight of the article.

20. A method of making up and/or caring for the nails and/or false nails, the method comprising at least one step consisting in applying at least one flexible article to a natural nail and/or a false nail, said flexible article comprising at least one adhesive layer, and being characterized in that said adhesive layer comprises at least:
two block copolymers;
a tackifier resin having a number-average molecular weight that is less than or equal to 10000 g/mol selected from rosin, rosin derivatives, hydrocarbon resins, and mixtures thereof; and
a plasticizer.

21. The article according to claim 2, wherein said at least one olefin comprises ethylene carbide monomers having one or two ethylenically unsaturated bonds, having 2 to 5 carbon atoms.

22. The article according to claim 4, wherein the styrene content in a block copolymer is less than 35%.

23. The article according to claim 5, wherein the other block copolymer is a triblock copolymer.

24. The article according to claim 8, wherein rosin derivatives are selected from rosin esters resulting from glycerol or from pentaerythritol.

25. The article according to claim 8, wherein hydrocarbon resins are selected from hydrogenated resins selected from indene hydrocarbon resins, aliphatic pentadiene resins, mixed pentadiene and indene resins, diene resins from cyclopentadiene dimers, hydrogenated resins from the polymerization of pentadiene, diene resins from isoprene dimers, and mixtures thereof.

26. The article according to claim 10, wherein the plasticizer is selected from polyisobutenes.
